(19) United States
(12) Patent Application Publication
(10) Pub. No.: US 2009/0054369 A1
(43) Pub. Date: Feb. 26, 2009

(54) PHARMACEUTICAL COMPOSITIONS OF SILICON-CONTAINING SUBSTITUTED ADENOSINE NUCLEOSIDE AMIDE ANALOGS

(75) Inventor: Stephen Gately, Scottsdale, AZ (US)

Correspondence Address: DUANE MORRIS LLP - Chicago;IP DEPARTMENT, 190 South LaSalle Street, Suite 3700, CHICAGO, IL, 60603-3433, US

(73) Assignee: RND PHARMACEUTICALS, INC., Scottsdale, AZ (US)

(21) Appl. No.: 11/997,896

(22) PCT Filed: Aug. 2, 2006
(86) PCT No.: PCT/US06/30246

§ 371 Date: Jun. 3, 2008

Related U.S. Application Data
(60) Provisional application No. 60/705,043, filed on Aug. 3, 2005.

(51) Int. Cl. A61K 31/7076; C07H 19/16; A61P 35/00

(52) U.S. Cl. 514/45; 536/27.62

(57) ABSTRACT

Novel compositions of silicon-containing anti-metabolite compounds, their preparation and use in methods for treating cancer are described. The silyl group confers lipophilicity that can enhance the penetration of the compounds across the gut wall, cell membranes and blood brain barrier, thus improving therapeutic properties including bioavailability, metabolism, and/or pharmacokinetics. The triorganosilyl group provides compounds having improved pharmacokinetics and anti-tumor activity. The invention encompasses novel compounds, analogs, prodrugs and pharmaceutically acceptable salts thereof, pharmaceutical compositions and methods for treatment of diseases and other maladies or conditions involving cancer and the like. The subject invention also relates to processes for making such compounds as well as to intermediates useful in such processes.

### BACKGROUND OF THE INVENTION

The present invention relates generally to the preparation of novel compositions of silicon-containing (sila) anti-metabolite compounds and derivatives, and the methods of use to achieve an enhanced antitumorigenic effect in mammals, particularly humans.

An anti-metabolite is a compound that has a similar structure to a metabolite, but competes with or replaces the metabolite, thus preventing or reducing its normal function in cellular metabolism. Anti-metabolites used in cancer therapy include methotrexate, analogues of nucleic acid bases such as mercaptopurine (Purinethol), azathioprine (Imuran), thioguanine, dacarbazine, nucleoside analogues, analogues of sugars such as mitobronitol, and analogues of steroid synthesis in the adrenals (e.g. mitotane), for example. Some anti-metabolites are also used to treat other proliferative diseases such as inflammatory conditions. Other examples of launched anti-cancer drugs that are nucleoside analogues include gemcitabine, 5-fluorouracil, fludarabine clofarabine and proguanil.

Nucleoside analogues are metabolized inside the cell to their phosphate that inhibits DNA elongation and repair via alpha-DNA polymerase, inhibition of DNA repair via an effect on beta-DNA polymerase, incorporation into DNA, decrease in the endogenous ATP pool, and at the nuclear level, they inhibit RNA synthesis.

Patent number WO2004/050666 A1 discloses silicon-containing (sila) analogues of cytarabine. These analogues result from having one or more silicon-based protecting groups bound at an oxygen atom, forming an-O—Si-linkage. The O—Si bond hydrolyses too readily, including sterically hindered O-triisopropyl silanes and O-tert-butyl trimethylsilanes suggesting these analogues will be too rapidly hydrolyzed to the parent compound. Chiu et al generated a series of N- and O-triorganosilyated agents, but again the N—Si and O—Si linkage is too readily hydrolyzed (Chiu F T, Chang Y H, Ozkan G, Zon G, Fichter K C, Phillips L R J Pharm Sci. 1982 71(5):542-551). Millership et al generated silyl derivatives of testoterone (Millership J S, Shanks M L J Pharm Sci. 1988 77(2):116-9). These testosteroxysilanes formed dimmers through the formation of silyl ethers (O—Si—O), yet even with the steric bulk of the steroid nucleus, the O—Si bond was rapidly hydrolyzed.

Taken together, these data demonstrate that new silicon compositions and methods of synthesis for sila-analogues of anti-metabolite compounds are needed. N-(trimethylsilylethoxycarbonyl)-deoxycitidine and deoxyadenosine derivatives have been synthesized and are reported to be stable intermediates for DNA synthesis. (see Sekine, M; To be, M; Nagayama, T; Wada T, Letters in Organic Chemistry, 2004, 1: 179-182). However, stable intermediates are still needed for use as pharmaceutical or therapeutic agents. It is therefore desirable to generate certain nucleoside analogs that contain silicon atom(s) within the molecule that have improved biological properties, and improved pharmacokinetics. It will also be important that these molecules retain the antitumorigenic effect of the parent drug, and yet exhibit other effects not exhibited by the drug prior to derivatization.

Silicon-substitution of drugs is an approach for generating organosilicon compounds that have beneficial biological properties. The approach involves the replacement of specific atoms in compounds with silicon, and monitoring how the biological properties of the compounds are altered. A review of this approach is provided in Tacke and Zilch, Endeavour, New Series, 10, 191-197 (1986); and Showell, G A and Mills, J S, Chemistry challenges in lead optimization: silicon isosteres in drug discovery. Drug Discovery Today 8(12): 551-556, 2003.

### SUMMARY AND OBJECTS OF THE INVENTION

The present invention is based on the discovery that lipophilic therapeutic agents can be generated following the straightforward reaction of various adenine nucleoside analogs with various triorganosilyl-substituted chlorosilane derivatives. These new analogs incorporate silicon atoms bound to carbon and provide products that resist hydrolysis and are useful in the treatment of many diseases, including diseases treated by their unsilylated counterpart molecules.

### DETAILED DESCRIPTION OF THE INVENTION

The invention concerns a particular class of silyl-containing compounds of formula I. The silyl group confers lipophilicity that can enhance the penetration of the compounds across the gut wall, cell membranes and blood brain barrier, thus improving therapeutic properties including bioavailability, metabolism, and/or pharmacokinetics. The triorganosilyl group provides compounds having improved pharmacokinetics and anti-tumor activity.

The present invention provides compounds incorporating silicon atom(s) that demonstrate enhanced pharmaceutical properties.

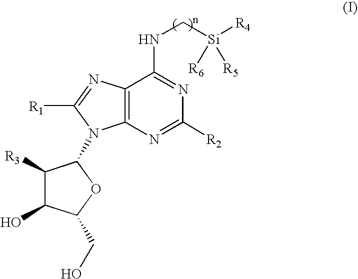

wherein

### R₁ can be H, F, Cl, Br or NH₂;

### R₂ can be H, F, Cl, Br;

### R₃ can be H, OH, F, Cl, Br;

n can be any integral valve that produces an active compound, preferably 1-6;
R₄, R₅, R₆ can be any group that does not substantially interfere with compound formation. Each R can be the same or different and can include, by way of example, methyl, ethyl, n-propyl, iso-propyl, n-butyl, iso-butyl, sec-butyl, tert-butyl, —CH₂CH(CH₂ CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and the like.

In another embodiment, the invention relates to a pharmaceutical composition including an effective amount of at least one compound of formula (I). The pharmaceutical composition has excellent pharmacokinetic profiles for treating tumors with high safety margin.

In another embodiment, the invention relates to a method of achieving an enhanced antitumorigenic effect in mammals, particularly humans.

In yet another embodiment, the invention relates to processes for producing derivatives of formula (I) that can be obtained by reacting a compound of formula (II) with a compound of formula (III) to generate stable silyl amides having at least one carbon spacer between the nitrogen and triorganosilyl moiety.

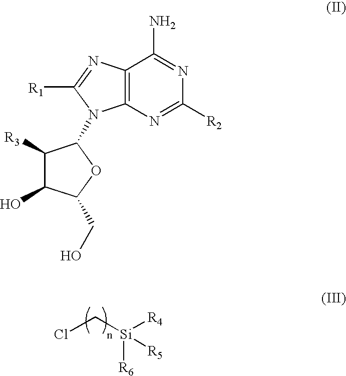

Preferred silicon derivatives of formula (III) include, but are not limited to, chloromethyltrimethylsilane, chloromethyldimethylpentylsilane, 3-chloropropyltrimethylsilane, 1-(trimethylsilyl)-4-chloro-3-methylbenzene, chloromethyldimethylphenylsilane, chloropropyldiphenylmethylsilane, 2-chloroethylmethyldimethoxysilane, 2-chloroethyltrimethoxysilane, 3-chloroisobutyldimethylmethoxysilane, chloromethyldimethylethoxysilane, chloromethylmethyldiethoxysilane, chloromethylmethyldiisopropoxysilane, chloromethylpentamethyldisiloxane, ((chloromethyl)phenethyl)-methyldimethoxysilane, (p-chloromethyl)phenyltrimethoxysilane, chloromethyltriethoxysilane, chloroethyltrimethoxysilane, 3-chloropropyldimethyldimethoxysilane, 3-chloropropylmethylmethoxysilane, 3-chloropropyltriethoxysilane, 3-chloropropyltrimethoxysilane, 3-chloropropyltris(trimethylsiloxy)silane.

Preferred silicon derivatives that can be generated using such methods and that are generally useful in the treatment of malignancies and other diseases include the following:
2-(8-chloro-6-((trimethylsilyl)methylamino)-9H-purin-9-yl)-5-(hydroxymethyl)-tetrahydrofuran-3,4-diol,
2-(8-chloro-6-((dimethyl(pentyl)silyl)methylamino)-9H-purin-9-yl)-5-(hydroxymethyl)-tetrahydrofuran-3,4-diol,
2-(8-chloro-6-(3-(trimethylsilyl)propylamino)-9H-purin-9-yl)-5-(hydroxymethyl)-tetrahydrofuran-3,4-diol,
2-(8-chloro-6-(2-methyl-4-(trimethylsilyl)phenylamino)-9H-purin-9-yl)-5-(hydroxymethyl)-tetrahydrofuran-3,4-diol,
2-(8-chloro-6-((dimethyl(phenyl)silyl)methylamino)-9H-purin-9-yl)-5-(hydroxymethyl)-tetrahydrofuran-3,4-diol,
2-(8-chloro-6-(3-(methyldiphenylsilyl)propylamino)-9H-purin-9-yl)-5-(hydroxymethyl)-tetrahydrofuran-3,4-diol,
2-(8-amino-6-((trimethylsilyl)methylamino)-9H-purin-9-yl)-5-(hydroxymethyl)-tetrahydrofuran-3,4-diol,
2-(8-amino-6-((dimethyl(pentyl)silyl)methylamino)-9H-purin-9-yl)-5-(hydroxymethyl)-tetrahydrofuran-3,4-diol,
2-(8-amino-6-(3-(trimethylsilyl)propylamino)-9H-purin-9-yl)-5-(hydroxymethyl)-tetrahydrofuran-3,4-diol,
2-(8-amino-6-(2-methyl-4-(trimethylsilyl)phenylamino)-9H-purin-9-yl)-5-(hydroxymethyl)-tetrahydrofuran-3,4-diol,
2-(8-amino-6-((dimethyl(phenyl)silyl)methylamino)-9H-purin-9-yl)-5-(hydroxymethyl)-tetrahydrofuran-3,4-diol,
2-(8-amino-6-(3-(methyldiphenylsilyl)propylamino)-9H-purin-9-yl)-5-(hydroxymethyl)-tetrahydrofuran-3,4-diol,
2-(2-fluoro-6-((trimethylsilyl)methylamino)-9H-purin-9-yl)-5-(hydroxymethyl)-tetrahydrofuran-3,4-diol,
2-(2-fluoro-6-((dimethyl(pentyl)silyl)methylamino)-9H-purin-9-yl)-5-(hydroxymethyl)-tetrahydrofuran-3,4-diol,
2-(2-fluoro-6-(3-(trimethylsilyl)propylamino)-9H-purin-9-yl)-5-(hydroxymethyl)-tetrahydrofuran-3,4-diol,
2-(2-fluoro-6-(2-methyl-4-(trimethylsilyl)phenylamino)-9H-purin-9-yl)-5-(hydroxymethyl)-tetrahydrofuran-3,4-diol,
2-(2-fluoro-6-((dimethyl(phenyl)silyl)methylamino)-9H-purin-9-yl)-5-(hydroxymethyl)-tetrahydrofuran-3,4-diol,
2-(2-fluoro-6-(3-(methyldiphenylsilyl)propylamino)-9H-purin-9-yl)-5-(hydroxymethyl)-tetrahydrofuran-3,4-diol,
5-(2-chloro-6-((trimethylsilyl)methylamino)-9H-purin-9-yl)-4-fluoro-2-(hydroxymethyl)-tetrahydrofuran-3-ol
5-(2-chloro-6-((dimethyl(pentyl)silyl)methylamino)-9H-purin-9-yl)-4-fluoro-2-(hydroxymethyl)-tetrahydrofuran-3-ol
5-(2-chloro-6-(3-(trimethylsilyl)propylamino)-9H-purin-9-yl)-4-fluoro-2-(hydroxymethyl)-tetrahydrofuran-3-ol
5-(2-chloro-6-(2-methyl-4-(trimethylsilyl)phenylamino)-9H-purin-9-yl)-4-fluoro-2-(hydroxymethyl)-tetrahydrofuran-3-ol
5-(2-chloro-6-((dimethyl(phenyl)silyl)methylamino)-9H-purin-9-yl)-4-fluoro-2-(hydroxymethyl)-tetrahydrofuran-3-ol
5-(2-chloro-6-(3-(methyldiphenylsilyl)propylamino)-9H-purin-9-yl)-4-fluoro-2-(hydroxymethyl)-tetrahydrofuran-3-ol
5-(2-chloro-6-((trimethylsilyl)methylamino)-9H-purin-9-yl)-2-(hydroxymethyl)-tetrahydrofuran-3-ol,
5-(2-chloro-6-((dimethyl(pentyl)silyl)methylamino)-9H-purin-9-yl)-2-(hydroxymethyl)-tetrahydrofuran-3-ol,
5-(2-chloro-6-(3-(trimethylsilyl)propylamino)-9H-purin-9-yl)-2-(hydroxymethyl)-tetrahydrofuran-3-ol,
5-(2-chloro-6-(2-methyl-4-(trimethylsilyl)phenylamino)-9H-purin-9-yl)-2-(hydroxymethyl)-tetrahydrofuran-3-ol,
5-(2-chloro-6-((dimethyl(phenyl)silyl)methylamino)-9H-purin-9-yl)-2-(hydroxymethyl)-tetrahydrofuran-3-ol,
5-(2-chloro-6-(3-(methyldiphenylsilyl)propylamino)-9H-purin-9-yl)-2-(hydroxymethyl)-tetrahydrofuran-3-ol, and

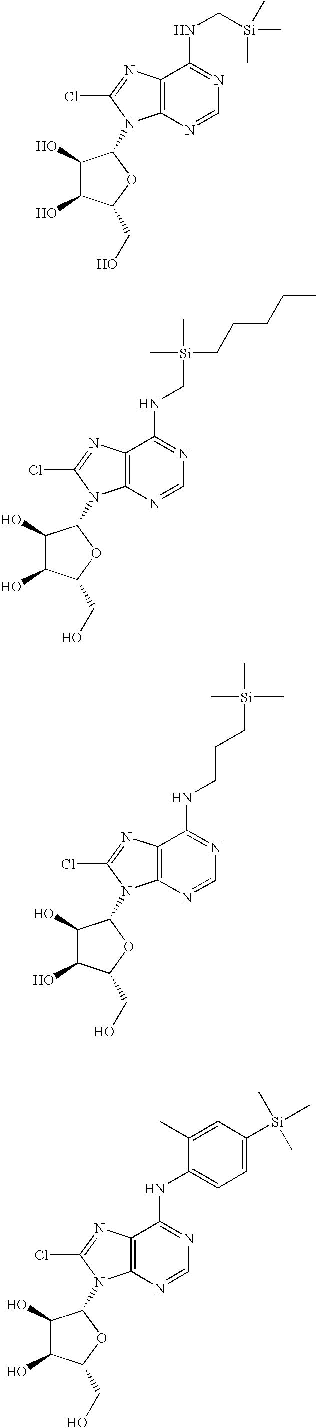

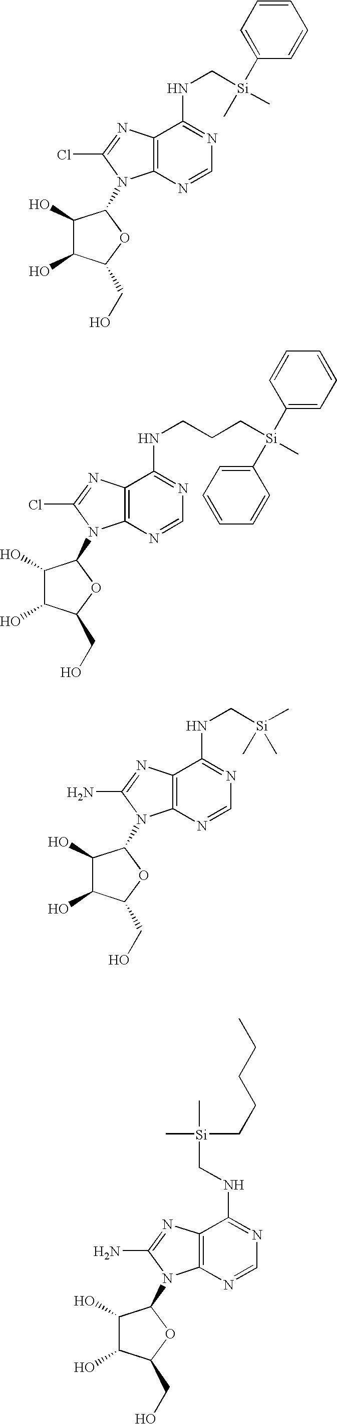

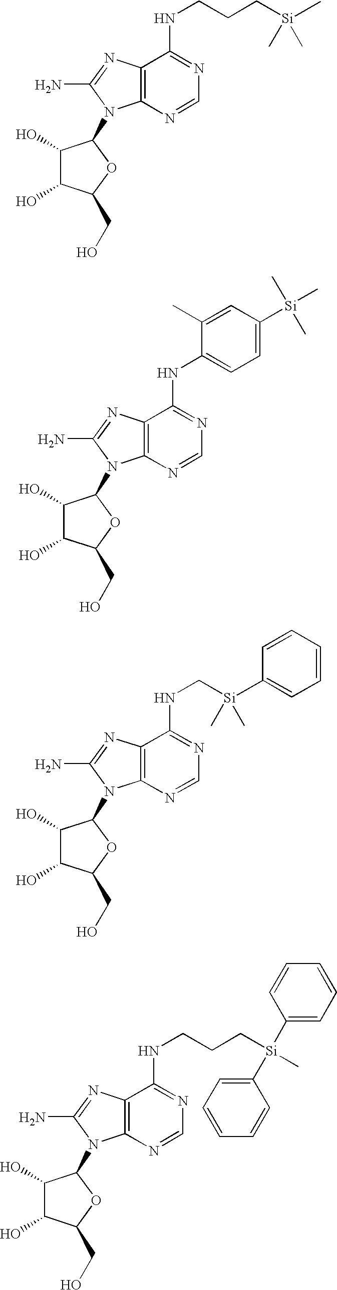

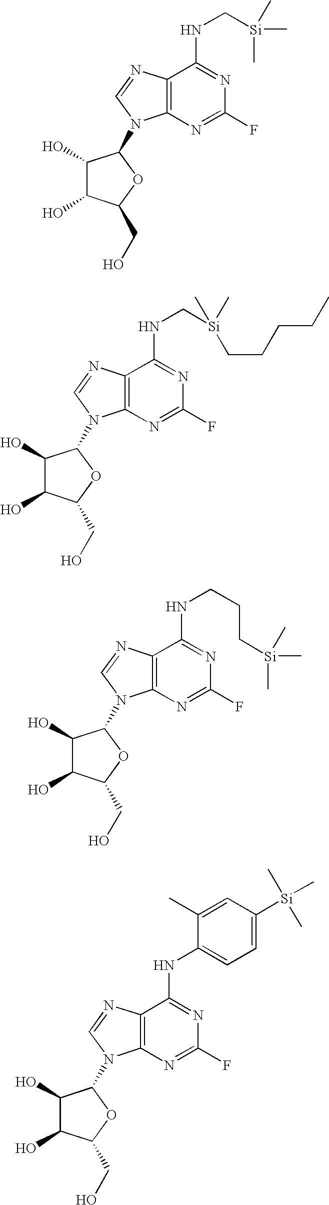

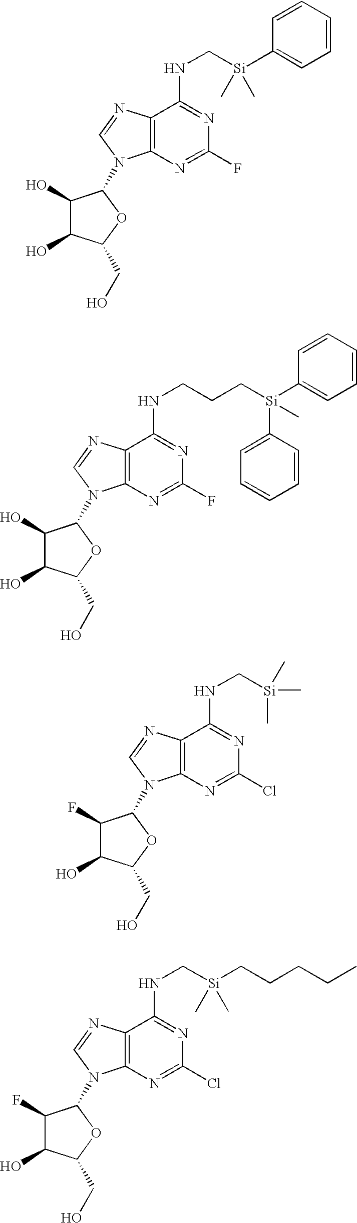

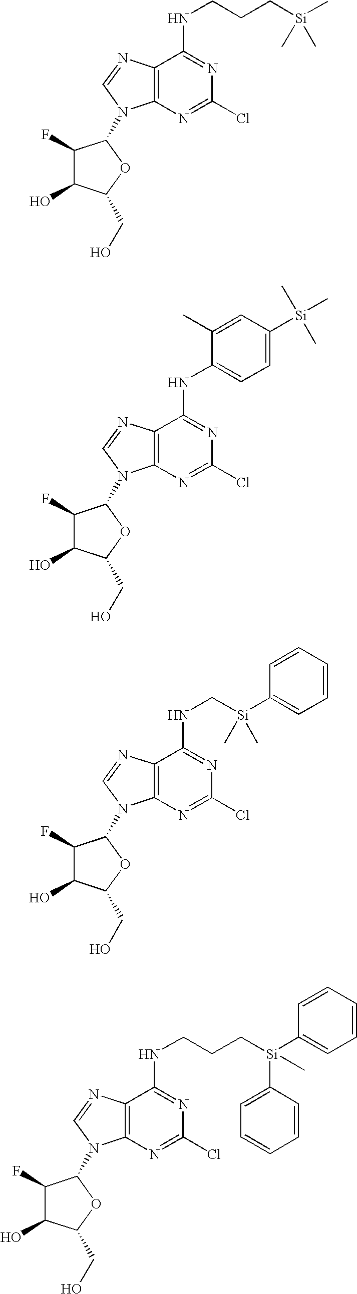

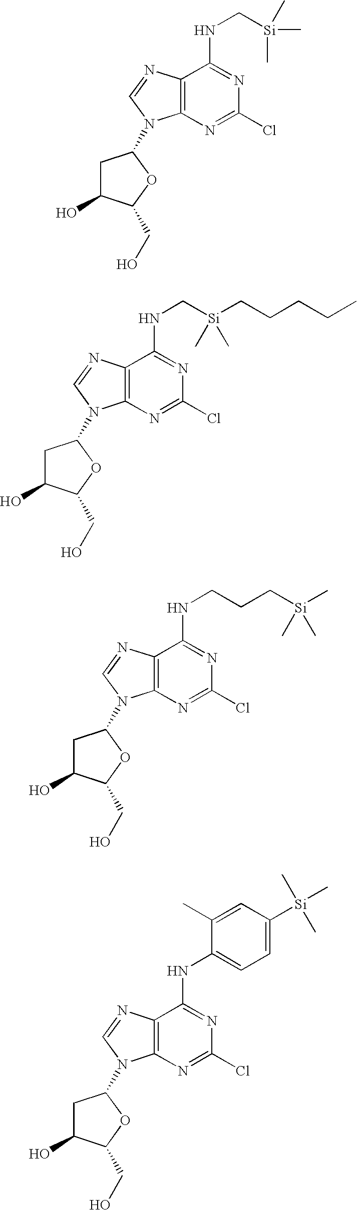

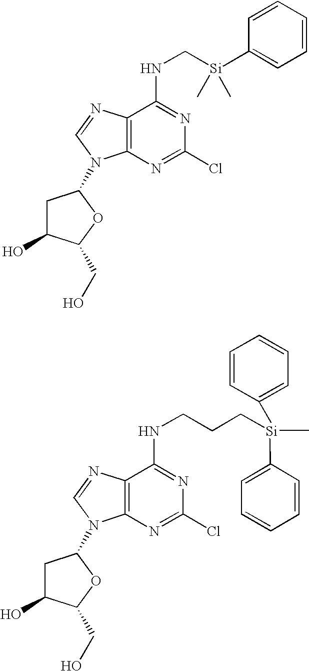

for example.

Compounds of particular interest in the treatment of malignancy, including hematologic malignancies, such as multiple myeloma and chronic lymphocytic leukemia (CLL) include sila derivatives of the 8-chloroadenosine or 8-aminoadenosine compounds. Thus, compounds having an R1 group that is chlorine or an amino group are also of particular interest.

Compounds of the invention may be chiral. They may be in the form of a single enantiomer or diastereomer, or a racemate. The stereochemistry of a chiral ring atom is preferably the same as that of the corresponding atom in the parent analog. More preferably, the stereochemistry of the compound as a whole corresponds to that of the parent molecule.

Compounds of the invention can be prepared in racemic form, or prepared in individual enantiomeric form by specific synthesis or resolution. The compounds may, for example, be resolved into their enantiomers by standard techniques, such as the formation of diastereomeric pairs by salt formation with an optically active acid followed by fractional crystallization and regeneration of the free base. Alternatively, the enantiomers of the novel compounds may be chromatographically separated, such as by HPLC, for example by using a chiral column, as is known.

Some compounds of the formula may exist in the form of various solvates, such as hydrates and also fall within the scope of the present invention.

Compounds of the invention may be in the form of pharmaceutically acceptable salts, for example, addition salts of inorganic or organic acids. Such inorganic acid addition salts include, for example, salts of hydrobromic acid, hydrochloric acid, nitric acid, phosphoric acid and sulphuric acid. Organic acid addition salts include, for example, salts of acetic acid, benzenesulphonic acid, benzoic acid, camphorsulphonic acid, citric acid, 2-(4-chlorophenoxy)-2-methylpropionic acid, 1,2-ethanedisulphonic acid, ethanesulphonic acid, ethylenediaminetetraacetic acid (EDTA), fumaric acid, glucoheptonic acid, gluconic acid, glutamic acid, N-glycolylarsanilic acid, 4-hexylresorcinol, hippuric acid, 2-(4-hydroxybenzoyl)benzoic acid, 1-hydroxy-2-naphthoic acid, 3-hydroxy-2-naphthoic acid, 2-hydroxyethanesulphonic acid, lactobionic acid, n-dodecyl sulphuric acid, maleic acid, malic acid, mandelic acid, methanesulphonic acid, methyl sulpuric acid, mucic acid, 2-naphthalenesulphonic acid, pamoic acid, pantothenic acid, phosphanilic acid ((4-aminophenyl) phosphonic acid), picric acid, salicylic acid, stearic acid, succinic acid, tannic acid, tartaric acid, terephthalic acid, p-toluenesulphonic acid, 10-undecenoic acid and the like.

It will be appreciated that such salts, provided that they are pharmaceutically acceptable, may be used in therapy. Such salts may be prepared by reacting the compound with a suitable acid in a conventional manner.

A compound of the invention may be prepared by any suitable method known in the art. Mixtures of final products or intermediates obtained can be separated on the basis of the physical-chemical differences of the constituents, by known methods, into the pure final products or intermediates, for example by chromatography, distillation, fractional crystallization, or by formation of a salt if appropriate or possible under the circumstances.

The preparation of the compounds of formula I are affected by a variety of procedures depending primarily upon the specific definitions of the n and R₁, R₂, R₃, R₄, R₅ and R₆ moieties. One of skill can appreciate that chemical reactions and procedures analogous to those known in the art and the selection of a particular route to obtain particular compounds is governed by known principles and can be obtained using methods that are analogous to the disclosed methods.

### Compound Preparation

The following routes of synthesis will serve to teach those of ordinary skill in the art how the compounds of formula I may be prepared.

The acetylation of a compound of formula (II) with acetic anhydride in dry pyridine gives an acetylated compound that is condensed with a substituted chlorosilane of formula (III) by means of pyridine in dichloromethane. Finally the compound is deacetylated with NaOH in dichloromethane/water as shown in synthetic scheme I.

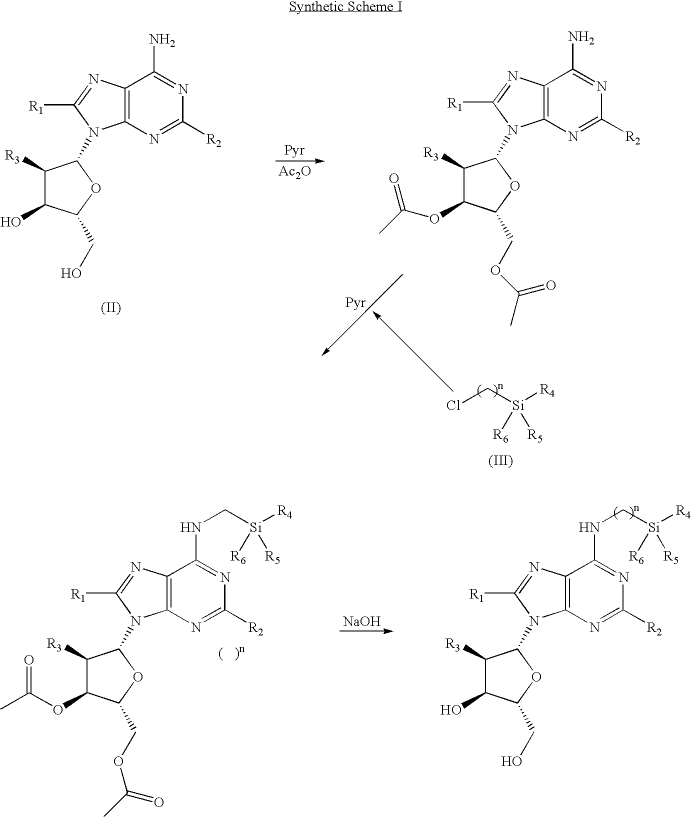

An alternative strategy using a transient protection strategy using trimethylsilyl chloride could be used. A compound of formula (II) is allowed to react with excess (6.0 molar equiv) of trimethylsilyl chloride in pyridine and the resulting mixture treated with 2 molar equivalent of a compound of formula (III). Treatment of the resulting product with 3% dichloracetic acid in CH₂Cl₂-MeOH [1:1 (v/v)] gave the desired product as shown in synthetic scheme II.

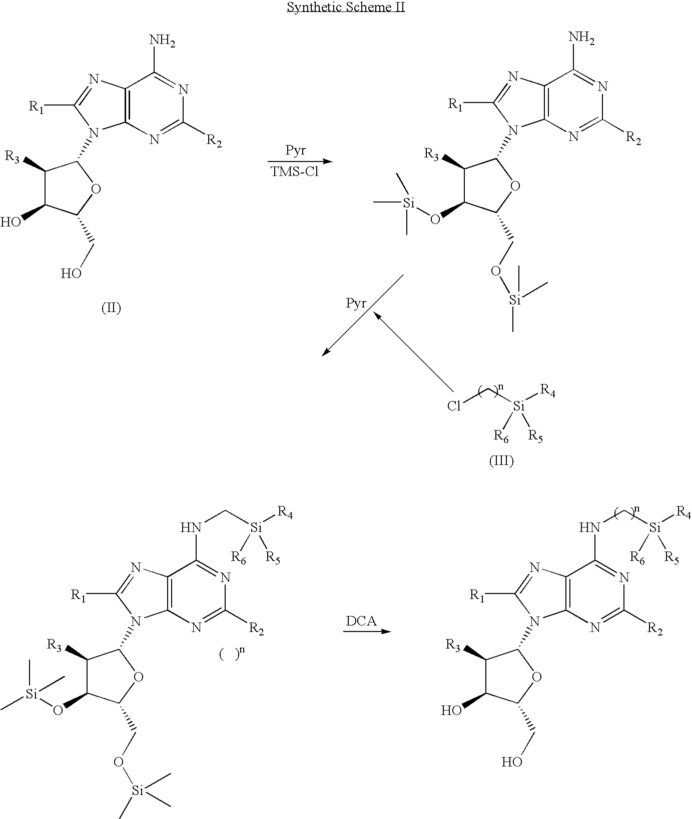

Another strategy to synthesize to synthesize compounds of the invention is represented in Scheme III. A compound of formula IV, with or without protection of sugar hydroxyl groups, is reacted with compounds of formula V in the presence of BOP and DIPEA in DMF, leading to the formation of N⁶-silaadenosine derivatives in good to excellent yields.

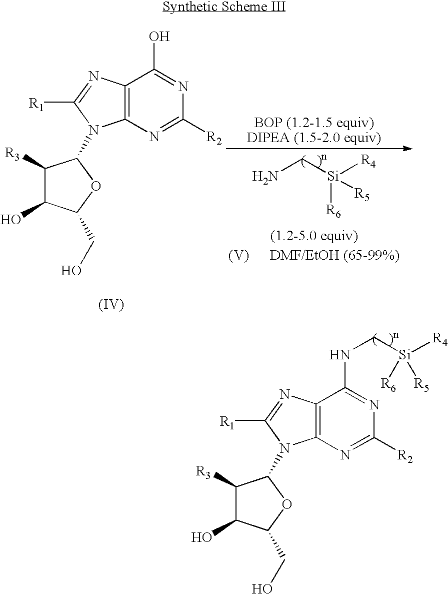

wherein

### R₁, can be H, F, Cl, Br or NH₂;

### R₂ can be H, F, Cl, Br;

### R₃ can be H, OH, F, Cl, Br;

n can be any integral valve that produces an active compound, preferably 1-6;
R₄, R₅, R₆ can be any group that does not substantially interfere with compound formation. Each R can be the same or different and can include, by way of example, methyl, ethyl, n-propyl, iso-propyl, n-butyl, iso-butyl, sec-butyl, tert-butyl, —CH₂CH(CH₂ CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and the like.

Preferred silicon derivatives include, aminomethyltrimethylsilane, aminopropyltrimethylsilane, (dimethyl(propyl)silyl)methanamine, aminobutyltrimethylsilane, (butyldimethylsilyl)methanamine, aminopentyltrimethylsilane, (dimethyl(pentyl)silyl)methanamine, aminohexyltrimethylsilane, (dimethyl(hexyl)silyl)methanamine, aminoheptyltrimethylsilane, (dimethyl(heptyl)silyl)methanamine, 1,1-dimethylsilinan-3-amine, 4-trimethylsilylaniline, (4-trimethylsilyl)phenyl)methanamine, 4-((trimethylsilyl)methyl)benzamine, 2-trimethylsilyl-5-aminopyridine, (dimethyl(pyridin-3-yl)silyl)methanamine, 2-(dimethyl(pyridin-3-yl)silyl)ethanamine, (dimethyl(phenyl)silyl)-methanamine, ((4-fluorophenyl)dimethylsilyl)methanamine, ((4-chlorophenyl)dimethylsilyl)methanamine, ((4-methoxyphenyl)dimethylsilyl)methanamine, (dimethyl(phenyl)silyl)-ethanamine, ((4-fluorophenyl)dimethylsilyl)ethanamine, ((4-chlorophenyl)dimethylsilyl)ethanamine, ((4-methoxyphenyl)dimethylsilyl)ethanamine.

Certain compounds could be used for the treatment of angiogenesis-related disorders in a subject. In a method the compounds can be administered to a subject in need of angiogenesis inhibition. The compounds could be used in the treatment of neoplasia, including metastasis; opthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

Certain compounds of the invention can be used for the prevention and treatment of benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. Certain compounds of the invention can be used for the prevention and treatment of hematologic malignancies, including but not limited to childhood leukemia, childhood lymphomas, multiple myeloma, Hodgkin's disease, lymphomas of lymphocytic origin, lymphomas of cutaneous origin, acute leukemia, chronic leukemia, acute lymphoblastic leukemia, acute myelocytic leukemia, chronic myelocytic leukemia, plasma cell neoplasm, lymphoid neoplasm and cancers associated with AIDS. Preferably, neoplasia is selected from gastrointestinal cancer, Barrett's esophagus, liver cancer, bladder cancer, pancreas cancer, ovary cancer, prostate cancer, cervical cancer, lung cancer, breast cancer and skin cancer, such as squamous cell and basal cell cancers, hematologic malignancies, childhood leukemia, childhood lymphomas, multiple myeloma, Hodgkin's disease, lymphomas of lymphocytic origin, lymphomas of cutaneous origin, acute leukemia, chronic leukemia, acute lymphoblastic leukemia, acute myelocytic leukemia, chronic myelocytic leukemia, plasma cell neoplasm, lymphoid neoplasm and cancers associated with AIDS. Certain compounds can also be used to treat the fibrosis that occurs with radiation therapy. The method can be used to treat subjects having adenomatous polyps, including those with familial adenomatous polyposis (FAP). Additionally, the method can be used to prevent polyps from forming in patients at risk of FAP. Such conditions are exemplary in nature, and are in no way meant to limit the scope of the invention.

The compounds of the present invention may be administered alone or in conjunction with additional therapies known to those skilled in the art in the prevention or treatment of neoplasia. Alternatively, the compounds described herein may be used in conjunctive therapy. By way of example, the compounds may be administered alone or in conjunction with other antineoplastic agents or other growth inhibiting agents or other drugs or nutrients.

There are large numbers of antineoplastic agents available and in commercial use, in clinical evaluation and in pre-clinical development, which could be selected for treatment of neoplasia by combination drug chemotherapy. Such antineoplastic agents fall into several major categories, namely, antibiotic-type agents, alkylating agents, antimetabolite agents, hormonal agents, immunological agents, or interferon-type agents among other miscellaneous agents.

A first family of antineoplastic agents that may be used in combination with compounds of the present invention consists of antimetabolite-type antineoplastic agents. Suitable antimetabolite antineoplastic agents may be selected from the group consisting of 5-FU-fibrinogen, acanthifolic acid, aminothiadiazole, brequinar sodium, carmofur, cyclopentyl cytosine, cytarabine phosphate stearate, cytarabine conjugates, dezaguanine, dideoxycytidine, dideoxyguanosine, didox, doxifluridine, fazarabine, floxuridine, fludarabine phosphate, 5-fluorouracil, N-(2′-furanidyl)-5-fluorouracil, isopropyl pyrrolizine, methobenzaprim, methotrexate, norspermidine, pentostatin, piritrexim, plicamycin, thioguanine, tiazofurin, trimetrexate, tyrosine kinase inhibitors, tyrosine protein kinase inhibitors, UFT or uricytin.

A second family of antineoplastic agents that may be used in combination with compounds of the present invention consists of alkylating-type antineoplastic agents. Suitable alkylating-type antineoplastic agents may be selected from the group consisting of aldo-phosphamide analogues, altretamine, anaxirone, bestrabucil, budotitane, carboplatin, carmustine, chlorambucil, cisplatin, cyclophosphamide, cyplatate, diphenylspiromustine, diplatinum cytostatic, Erba distamycin derivatives, elmustine, estramustine phosphate sodium, fotemustine, hepsul-fam, ifosfamide, iproplatin, lomustine, mafosfamide, mitolactol, oxaliplatin, Upjohn PCNU, prednimustine, ranimustine, semustine, spiromus-tine, tauromustine, temozolomide, teroxirone, tetraplatin or trimelamol.

A third family of antineoplastic agents which may be used in combination with compounds of the present invention consists of antibiotic-type antineoplastic agents. Suitable antibiotic-type antineoplastic agents may be selected from the group consisting of aclarubicin, actinomycin D, actinoplanone, aeroplysinin derivative, anthracycline, azino-mycin-A, bisucaberin, bleomycin sulfate, bryostatin-1, calichemycin, chromoximycin, dactinomycin, daunorubicin, ditrisarubicin B, doxorubicin, doxorubicin-fibrinogen, elsamicin-A, epirubicin, erbstatin, esorubicin, esperamicin-A1, esperamicin-Alb, fostriecin, glidobactin, gregatin-A, grincamycin, herbimycin, idarubicin, illudins, kazusamycin, kesarirhodins, menogaril, mitomycin, mitoxantrone, neoenactin, oxalysine, oxaunomycin, peplomycin, pilatin, pirarubicin, porothramycin, pyrindamycin A, rapamycin, rhizoxin, rodorubicin, sibanomicin, siwenmycin, sorangicin-A, sparsomycin, steffimycin B, talisomycin, terpentecin, thrazine, tricrozarin A, or zorubicin.

A fourth family of antineoplastic agents which may be used in combination with compounds of the present invention consists of a miscellaneous family of antineoplastic agents selected from the group consisting of alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristo-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, elliprabin, elliptinium acetate, Tsumura EPMTC, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanine derivatives, methylanilinoacridine, Molecular Genetics MGI-136, minactivin, mitonafide, mitoquidone, mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, octreotide, Ono ONO-112, oquizanocine, Akzo Org-10172, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin 1, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, with anolides or Yamanouchi YM-534.

A fifth family of antineoplastic agents can be used in combination with compounds of the present invention including kinase inhibitors, such as tyrosine kinase inhibitors, particularly including but not limited to, Herceptin, erbitux, imatinib (Gleevec), dasatinib (BMS-354825).

In addition, the disclosed antineoplastic agents can be used in combination with supportive care agents. For example, patients can be treated with 360-480 Units of GCSF daily, or receive a long acting form of GCSF, such as Neulasta in combination with compounds of the present invention.

Additionally, erythropoietin can be administered at least once during one of the weeks the compounds of this invention is administered. In a method, patients could receive in conjunction with one or more of the disclosed compounds of the invention about 40,000 Units of erythropoietin. Suitable formulations of erythropoietin include Epogen™ and ProQuist™ formulations or long-acting formulations such as Aranist™ formulations.

Examples of radioprotective agents which may be used in combination with compounds of the present invention are AD-5, adchnon, amifostine analogues, detox, dimesna, 1-102, MM-159, N-acylated-dehydroalanines, TGF-Genentech, tiprotimod, amifostine, WR-151327, FUT-187, ketoprofen transdermal, nabumetone, superoxide dismutase.

The methods and combinations of the present invention may be used for the treatment of neoplasia disorders selected from the group consisting of acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondrosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiatied carcinoma, uveal melanoma, verrucous carcinoma, vipoma, well differentiated carcinoma, or Wilm's tumor.

Besides being useful for human treatment, these compounds are also useful for veterinary treatment of companion animals, exotic animals or farm animals, including mammals, rodents, or the like. More preferred animals include horses, dogs, or cats.

The present compounds may also be used in co-therapies, partially or completely, such as together with steroids, NSAIDs, iNOS inhibitors, p-38 inhibitors, TNF inhibitors, 5-lipoxygenase inhibitors, LTB₄ receptor antagonists or LTA₄ hydrolase inhibitors.

The pharmaceutical composition may include, but is not limited to, at least one acceptable carrier. The carrier can generally be an inert bulk agent added to make the active ingredients easier to handle, and can be solid, semisolid, or liquid, as well as any pharmaceutically acceptable carrier known in the art that does not interfere with stability or activity of the active agent(s).

Pharmaceutical compositions of the invention may also include any delivery vehicle or device known in the art to enhance transport in reaching the target site. Such delivery vehicles or devices may be administered in admixtures with any carrier aforementioned with regard to the route of administration, and standard pharmaceutical practice. Dosages associated with such vehicles or devices will vary according to certain factors, such as age, weight, and the condition of the human or animal, as well as the pharmacokinetics and release characteristics from said delivery vehicles or devices.

The present invention also involves a method for the treatment of the conditions as noted above in mammals, particularly humans, suffering therefrom.

The present invention also provides for the use of any such compound of the above pharmaceutical compositions or salt thereof, in the manufacture of a therapeutic agent.

Treatment is contemplated in mammals, particularly humans, as well as those mammals of economic or social importance, or of an endangered status. Examples may be livestock or other animals expressly for human consumption, or domesticated animals such as dogs, cats, or horses. Also contemplated is the treatment of birds or poultry, such as turkeys, chickens, or fowl or the like.

The invention comprises administration of a treatment-effective amount of silicon-containing anti-metabolite derivatives in concentrations calculated to provide the mammal being treated in the prevention, control, or cessation of disease.

The mode of administration of the silicon-containing anti-metabolite compound to the human or animal to be treated is as such to deliver an inhibiting effective amount of the pharmaceutical composition. For example, therapeutic delivery may be achieved, but is not limited to, enteral administration, which includes oral, sublingual, or rectal administration or via parenteral administration that includes intramuscular, intravenous, or subcutaneous. Therapeutic delivery may also be achieved via other routes including topical, transdermal, or inhalation. Formulations of the compounds may include, but are not limited to, transdermal patch, suppository, tablet, capsule, powder, or in an appropriate carrier fluid administered in a solution or suspension. Also contemplated is administration of the solution or suspension to the esophagus, stomach, and/or duodenum, such as by gavage, i.e., via a feeding tube.

It is also contemplated that additional ingredients, such as various excipients, carriers, surfactants, nutriments, and the like, as well as various medicaments other than a sila-anti-metabolite derivative, or combinations thereof, may be present together with the sila-anti-metabolite derivative.

A dosing amount of silicon-containing anti-metabolite derivatives suitable to be therapeutically effective in a mammal, including humans, is to be calculated according to mg/kg of body weight, or body surface area (BSA). Administration may be one or more times per day to achieve the total desired daily dose, the amount varying as to the severity of the cancer or condition as previously discussed.

The present invention may be useful in the treatment of cancer in humans or animals, wherein the cancer or condition is caused as a result of exposure to any number of pathogens; nutritional factors; environmental factors that act as stressors or pollutants; and/or physiological disorders such as those of the digestive tract, pulmonary/circulatory system, liver, kidneys, colon, and/or pancreas.

In addition to the foregoing, the present invention also contemplates a process for the production of a pharmaceutical composition. Such process comprises bringing at least one of the individual components described thereof into intimate admixture with a silicon-modified anti-metabolite of the present invention, and when required, compounding the obtained composition(s) in unit dosage forms. Methods of preparation of pharmaceutical compositions are not described here in detail, but are known in the art. For the discussion of such methods, pages 1435-1694 of Remington's Pharmaceutical Science (Part 8) are incorporated herein by reference.

The foregoing descriptions have been directed to particular embodiments of the invention in accordance with requirements of the Patent Statutes for the purposes of illustration and explanation. It will be apparent however, to those skilled in the art, that many modifications, changes and variations in the claimed compositions, solutions, methods of administration of the compositions set forth will be possible without departing from the scope and spirit of the claimed invention. It is intended that the following claims be interpreted to embrace all such modifications and changes.

We claim:

1. A compound of formula (I), 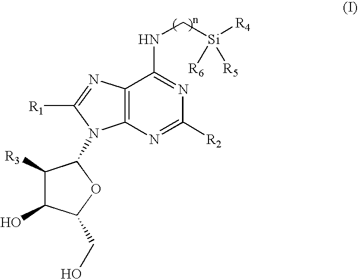
wherein
R₁ is H, F, Cl, Br or NH₂;
R₂ is H, F, Cl, Br;
R₃ is H, O, F, Cl, Br;
n is an integer;
and wherein R₄, R₅, R₆ can be the same or different and can be selected from the chemical groups consisting of methyl, ethyl, n-propyl, iso-propyl, n-butyl, iso-butyl, sec-butyl, tert-butyl, —CH₂CH(CH₂ CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and hydrates and solvates thereof.

2. The compounds according to claim 1, selected from a group consisting of:
2-(8-chloro-6-((trimethylsilyl)methylamino)-9H-purin-9-yl)-5-(hydroxymethyl)-tetrahydrofuran-3,4-diol,
2-(8-chloro-6-((dimethyl(pentyl)silyl)methylamino)-9H-purin-9-yl)-5-(hydroxymethyl)-tetrahydrofuran-3,4-diol,
2-(8-chloro-6-(3-(trimethylsilyl)propylamino)-9H-purin-9-yl)-5-(hydroxymethyl)-tetrahydrofuran-3,4-diol,
2-(8-chloro-6-(2-methyl-4-(trimethylsilyl)phenylamino)-9H-purin-9-yl)-5-(hydroxymethyl)-tetrahydrofuran-3,4-diol,
2-(8-chloro-6-((dimethyl(phenyl)silyl)methylamino)-9H-purin-9-yl)-5-(hydroxymethyl)-tetrahydrofuran-3,4-diol,
2-(8-chloro-6-(3-(methyldiphenylsilyl)propylamino)-9H-purin-9-yl)-5-(hydroxymethyl)-tetrahydrofuran-3,4-diol,
2-(8-amino-6-((trimethylsilyl)methylamino)-9H-purin-9-yl)-5-(hydroxymethyl)-tetrahydrofuran-3,4-diol,
2-(8-amino-6-((dimethyl(pentyl)silyl)methylamino)-9H-purin-9-yl)-5-(hydroxymethyl)-tetrahydrofuran-3,4-diol,
2-(8-amino-6-(3-(trimethylsilyl)propylamino)-9H-purin-9-yl)-5-(hydroxymethyl)-tetrahydrofuran-3,4-diol,
2-(8-amino-6-(2-methyl-4-(trimethylsilyl)phenylamino)-9H-purin-9-yl)-5-(hydroxymethyl)-tetrahydrofuran-3,4-diol,
2-(8-amino-6-((dimethyl(phenyl)silyl)methylamino)-9H-purin-9-yl)-5-(hydroxymethyl)-tetrahydrofuran-3,4-diol,
2-(8-amino-6-(3-(methyldiphenylsilyl)propylamino)-9H-purin-9-yl)-5-(hydroxymethyl)-tetrahydrofuran-3,4-diol,
2-(2-fluoro-6-((trimethylsilyl)methylamino)-9H-purin-9-yl)-5-(hydroxymethyl)-tetrahydrofuran-3,4-diol,
2-(2-fluoro-6-((dimethyl(pentyl)silyl)methylamino)-9H-purin-9-yl)-5-(hydroxymethyl)-tetrahydrofuran-3,4-diol,
2-(2-fluoro-6-(3-(trimethylsilyl)propylamino)-9H-purin-9-yl)-5-(hydroxymethyl)-tetrahydrofuran-3,4-diol,
2-(2-fluoro-6-(2-methyl-4-(trimethylsilyl)phenylamino)-9H-purin-9-yl)-5-(hydroxymethyl)-tetrahydrofuran-3,4-diol,
2-(2-fluoro-6-((dimethyl(phenyl)silyl)methylamino)-9H-purin-9-yl)-5-(hydroxymethyl)-tetrahydrofuran-3,4-diol,
2-(2-fluoro-6-(3-(methyldiphenylsilyl)propylamino)-9H-purin-9-yl)-5-(hydroxymethyl)-tetrahydrofuran-3,4-diol,
5-(2-chloro-6-((trimethylsilyl)methylamino)-9H-purin-9-yl)-4-fluoro-2-(hydroxymethyl)-tetrahydrofuran-3-ol
5-(2-chloro-6-((dimethyl(pentyl)silyl)methylamino)-9H-purin-9-yl)-4-fluoro-2-(hydroxymethyl)-tetrahydrofuran-3-ol
5-(2-chloro-6-(3-(trimethylsilyl)propylamino)-9H-purin-9-yl)-4-fluoro-2-(hydroxymethyl)-tetrahydrofuran-3-ol
5-(2-chloro-6-(2-methyl-4-(trimethylsilyl)phenylamino)-9H-purin-9-yl)-4-fluoro-2-(hydroxymethyl)-tetrahydrofuran-3-ol
5-(2-chloro-6-((dimethyl(phenyl)silyl)methylamino)-9H-purin-9-yl)-4-fluoro-2-(hydroxymethyl)-tetrahydrofuran-3-ol
5-(2-chloro-6-(3-(methyldiphenylsilyl)propylamino)-9H-purin-9-yl)-4-fluoro-2-(hydroxymethyl)-tetrahydrofuran-3-ol
5-(2-chloro-6-((trimethylsilyl)methylamino)-9H-purin-9-yl)-2-(hydroxymethyl)-tetrahydrofuran-3-ol,
5-(2-chloro-6-((dimethyl(pentyl)silyl)methylamino)-9H-purin-9-yl)-2-(hydroxymethyl)-tetrahydrofuran-3-ol,
5-(2-chloro-6-(3-(trimethylsilyl)propylamino)-9H-purin-9-yl)-2-(hydroxymethyl)-tetrahydrofuran-3-ol,
5-(2-chloro-6-(2-methyl-4-(trimethylsilyl)phenylamino)-9H-purin-9-yl)-2-(hydroxymethyl)-tetrahydrofuran-3-ol,
5-(2-chloro-6-((dimethyl(phenyl)silyl)methylamino)-9H-purin-9-yl)-2-(hydroxymethyl)-tetrahydrofuran-3-ol,
5-(2-chloro-6-(3-(methyldiphenylsilyl)propylamino)-9H-purin-9-yl)-2-(hydroxymethyl)-tetrahydrofuran-3-ol.

3. A pharmaceutical composition comprising a therapeutically effective amount of one or more compounds of formula (I) 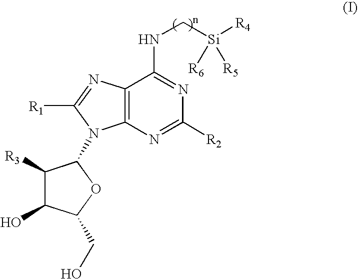
wherein
R₁ is H, F, Cl, Br or NH₂;
R₂ is H, F, Cl, Br;
R₃ is H, O, F, Cl, Br;
n is an integer;
and wherein R₄, R₅, R₆ can be the same or different and can include a group selected from the groups consisting of methyl, ethyl, n-propyl, iso-propyl, n-butyl, iso-butyl, sec-butyl, tert-butyl, —CH₂CH(CH₂ CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and hydrates and solvates thereof; and an inert carrier.

4. A method for treatment of a subject having a condition or disorder comprising administering to the subject a therapeutically effective amount of A compound of formula (I), 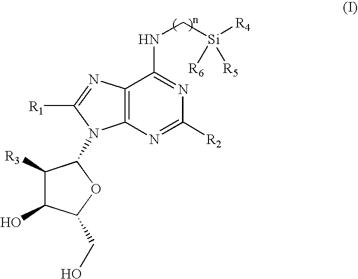
wherein
R₁ is H, F, Cl, Br or NH₂;
R₂ is H, F, Cl, Br;
R₃ is H, O, F, Cl, Br;
n is an integer;
and wherein R₄, R₅, R₆ can be the same or different and can be selected from the chemical groups consisting of methyl, ethyl, n-propyl, iso-propyl, n-butyl, iso-butyl, sec-butyl, tert-butyl, —CH₂CH(CH₂ CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and hydrates and solvates thereof.

5. The method of claim 4, in which the condition or disorder is angiogenesis.

6. The method of claim 4, in which the condition or disorder is cancer.

7. The method of claim 4, in which the condition or disorder is a hematologic malignancy.

8. The method of claim 4, in which the condition or disorder is the hematologic malignancy known as multiple myeloma.

9. The method of claim 4, in which the condition or disorder is the a hematologic malignancy chronic known as lymphocytic leukemia.

10. The method of claim 4, in which the condition or disorder is a hematologic malignancy and R₁ is an amino or chloride group.

11. The method of claim 4, wherein the subject is a mammal.

12. The method of claim 4, wherein the subject is human.

13. A method for treating a neoplasia in a subject in need of such treatment wherein the method comprises treating the subject with an amount of radiation and a radiation-potentiating amount of a compound of claim 1 or a pharmaceutically acceptable salt thereof wherein: the amount of radiation and the amount of the compound of claim 1 together comprise a neoplasia-treating-effective amount, and the neoplasia is sensitive to such treatment.

14. The compound of claim (1) wherein the compound is a diasteriomer, racemate, or single enantioner.

15. The pharmaceutical composition of claim 3 further comprising a second active agent.

16. The pharmaceutical composition of claim 3 further comprising a second active agent comprising an antineoplastic agent or growth inhibiting agent.

17. The pharmaceutical composition of claim 15 wherein the antineoplastic agent is an antimetabolite-type antineoplastic agent.

18. The pharmaceutical composition of claim 15 wherein the antineoplastic agent is an alkylating-type antineoplastic agent.

19. The pharmaceutical composition of claim 15 wherein the antineoplastic agent is an antibiotic-type antineoplastic agent.

20. The pharmaceutical composition of claim 15 wherein the antineoplastic agent is of antineoplastic agents selected from the group consisting of alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristo-Myers BMY-40481 Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, elliprabin, elliptinium acetate, Tsumura EPMTC, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanine derivatives, methylanilinoacridine, Molecular Genetics MGI-136, minactivin, mitonafide, mitoquidone, mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, octreotide, Ono ONO-112, oquizanocine, Akzo Org-10172, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, with anolides or Yamanouchi YM-534.

21. The pharmaceutical composition of claim 15 wherein the second active agent is a radioprotective protective agent.

22. The composition of claim 1 or claim 3 wherein n is 1-6.
